(19) United States
(12) Patent Application Publication
(10) Pub. No.: US 2026/0290554 A1
(43) Pub. Date: Sep. 24, 2026

(54) SYSTEM AND METHOD FOR NAVIGATING A TREATMENT PROCESS

(71) Applicant: Hazelden Betty Ford Foundation, Center City, MN (US)

(72) Inventor: Joseph S. Lee, Edina, MN (US)

(21) Appl. No.: 19/569,665

(22) Filed: Mar. 17, 2026

Related U.S. Application Data
(60) Provisional application No. 63/773,937, filed on Mar. 18, 2025.

(51) Int. Cl. G16H 20/70; G16H 70/20

(52) U.S. Cl. CPC G16H 20/70; G16H 70/20

(57) ABSTRACT

Systems and methods for navigating a treatment process are provided. Particularly, systems and methods for assisting patient-adjacent people to navigate a treatment process are provided. More particularly, systems and methods for assisting patient-adjacent people to navigate a treatment process by contextualizing information and providing it at timely junctures in the treatment process are provided. The method may include associating a patient-adjacent person with a patient, assessing the treatment process that the patient is undergoing, and developing a patient-adjacent person program. The treatment process may include segments, junctures, and/or milestones. The program provides content to the patient-adjacent person and contextualizes the content based on at least one of the segments, junctures, and/or milestones.

### CROSS-REFERENCE TO RELATED APPLICATIONS

This application claims the benefit of and priority to United States Provisional Patent Application Ser. No. 63/773,937 filed Mar. 18, 2025, the disclosure of which is herein incorporated by reference in its entirety.

### FIELD OF THE INVENTION

The present disclosure relates to novel and advantageous systems and methods for navigating a treatment process. Particularly, the present disclosure relates to novel and advantageous systems and methods for assisting patient-adjacent people to navigate a treatment process. More particularly, the present disclosure relates to novel and advantageous systems and methods for assisting patient-adjacent people to navigate a treatment process by contextualizing information and providing it at timely junctures in the treatment process.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Families, caregivers and loved ones (referred to herein as “patient-adjacent people”, or a “patient-adjacent person”) are an essential part of the treatment process, and their involvement has been linked to improved patient outcomes, as substance use disorder can pose a negative impact to an entire family system. Patient-adjacent people often carry a heavy burden. Research shows that engaged caregivers make a difference in the treatment outcomes of their loved ones and the engagement of caregivers highlights the importance of their own quality of life.

There are a number of situations where it may be useful for a person to enter a treatment program. Such treatment program may be for behavioral health, mental health, physical health, personal relationships, employment training, or other.

With specific reference to behavioral health, it is common for a patient to go through a relatively predictable treatment process. A plurality of resources are available to the patient as they go through that process. People adjacent to the patient, such as family members, are generally left on their own or are given one or more books with “how to cope” tips.

The resources provided to patient-adjacent persons, including books and the like, typically lack guidance on what behavior or emotional experiences may be normal or abnormal from a patient at what stages in the treatment process. Or, if any of that guidance is given, provides the information in bulk, requiring the patient-adjacent person to parse it and apply it at the relevant time independently. More particularly, these books do not provide step by step guidance tailored to what a patient is going through at that time and what the patient-adjacent people can expect. Nor do they provide a means by which the patient-adjacent people can input their experience to tailor the advice given. The patient-adjacent people have no context or grounding through the stages. There is no context for saying if what the patient or the patient-adjacent person is experiencing is normal, pathological, etc. There is very little guidance for patient-adjacent people for when they are experiencing the emotions and it can be difficult to find others who can understand what they are experiencing. Nothing saying “this is right sized, here is some help for you.”

Thus, there is a need in the art for systems and methods for assisting patient-adjacent people to navigate a treatment process. More particularly, there is a need for systems and methods for assisting patient-adjacent people to navigate a treatment process by contextualizing information and providing it at timely junctures throughout the treatment process.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present invention offers intensive programming resources both for a patient and a patient-adjacent person, such as a caregiver or family member, at meaningful junctures to the corresponding patient treatment process. References herein to participants may refer to the patient, the patient adjacent person, or both. The invention uses curated data sets and known common and important junctures where the patient-adjacent person may benefit from knowledge about the process, to anticipate and validate their experiences without requiring explicit requests for it. This forges a powerful sense of trust and understanding, which in turn solidifies the rapport needed to improve their own quality of life while better supporting their loved one in care.

In one embodiment, the present disclosure provides a method for guiding a patient-adjacent person through a treatment process, which may be a substance abuse disorder (“SUD”) or other behavioral health treatment process. The method includes associating a patient-adjacent person with a patient; assessing the treatment process that the patient is undergoing, and developing a patient-adjacent person program. The treatment process includes segments, junctures, and/or milestones. The program provides content to the patient-adjacent person and the contextualizes the content based on at least one of the segments, junctures, and/or milestones. In some embodiments, the developed patient-adjacent person program has an initial configuration and is revised to a modified configuration as the patient proceeds through treatment.

In another aspect, the present disclosure provides a system for guiding a patient-adjacent person through a treatment process, the system comprising: a processor; and memory storing instructions which, when executed by the processor, cause the system to: associate a patient-adjacent person with a patient; assess the treatment process that the patient is undergoing, wherein the treatment process includes at least one of segments, junctures, and milestones; and access a patient-adjacent person program based on the treatment process, wherein the program provides content to the patient-adjacent person. The patient-adjacent person program contextualizes the content based on at least one of the segments, junctures, and milestones. In some embodiments, the patient-adjacent person program has an initial configuration and is revised to a modified configuration as the patient proceeds through treatment, based on actions of at least one of the patient and the patient-adjacent person.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1a illustrates a timeline showing patient navigation through a treatment process and patient-adjacent person navigation through a treatment process, in accordance with one embodiment.

FIG. 1b illustrates admission and initial configuration set up of the timeline of FIG. 1a.

FIG. 1c illustrates treatment and alignment of a patient-adjacent person program of the timeline of FIG. 1a.

FIG. 2 illustrates a daily welcome page, in accordance with one embodiment.

FIG. 3 illustrates a self-tracker page, in accordance with one embodiment.

FIG. 4 illustrates a cover page of a Family Journey, in accordance with one embodiment.

FIG. 5 illustrates a welcome landing screen for a participant in a Family Journey, in accordance with embodiments of the present disclosure.

FIG. 6 illustrates a subsequent preparatory screen for getting started in a Family Journey, in accordance with embodiments of the present disclosure.

FIG. 7 illustrates an informational screen for participants in a Family Journey to understand the basics of SUDs, in accordance with embodiments of the present disclosure.

FIG. 8 illustrates an information and access screen for participants in a Family Journey to understand the importance of family involvement, in accordance with embodiments of the present disclosure.

FIG. 9 illustrates an information and access screen for participants in a Family Journey to support a loved one early in treatment, in accordance with embodiments of the present disclosure.

FIG. 10 illustrates an information and access screen to remind participants in a Family Journey to take personal care, in accordance with embodiments of the present disclosure.

FIG. 11 illustrates an information and access screen to remind participants in a Family Journey to find calm, in accordance with embodiments of the present disclosure.

FIG. 12 illustrates an information and access screen for participants in a Family Journey to understand strong emotions related to treatment, in accordance with embodiments of the present disclosure.

FIG. 13 illustrates an information and access screen to encourage participants in a Family Journey to seek advice from others facing similar circumstances, in accordance with embodiments of the present disclosure.

FIG. 14 illustrates an information and access screen for participants in a Family Journey to explore the family impacts of addiction, in accordance with embodiments of the present disclosure.

FIG. 15 illustrates an information and access screen to offer encouragement to participants in a Family Journey from professionals in charge of treatment aspects, in accordance with embodiments of the present disclosure.

FIG. 16 illustrates an information and access screen to encourage participants in a Family Journey to practice self care, in accordance with embodiments of the present disclosure.

FIG. 17 illustrates an information and access screen to encourage participants in a Family Journey to offer hope in SUD recovery, in accordance with embodiments of the present disclosure.

FIG. 18 illustrates an information and access screen for participants in a Family Journey to understand strong emotions related to treatment, in accordance with embodiments of the present disclosure.

FIG. 19 illustrates an information and access screen for participants in a Family Journey to explore the Seven Cs (cause, control, cure, care, communicate, choice, and celebrate) of SUDs, in accordance with embodiments of the present disclosure.

FIG. 20 illustrates an information and access screen for participants in a Family Journey to understand the role of self-esteem in SUD treatment, in accordance with embodiments of the present disclosure.

FIG. 21 illustrates an information and access screen to encourage participants in a Family Journey to practice mindfulness, in accordance with embodiments of the present disclosure.

FIG. 22 illustrates an information and access screen to encourage participants in a Family Journey to reassess trauma response and unhelpful thinking patterns, in accordance with embodiments of the present disclosure.

FIG. 23 illustrates an information and access screen for participants in a Family Journey to understand strong emotions of shame and guilt related to treatment, in accordance with embodiments of the present disclosure.

FIG. 24 illustrates an information and access screen to encourage participants in a Family Journey to sustain and improve healthy communication practices, in accordance with embodiments of the present disclosure.

FIG. 25 illustrates an information and access screen for participants in a Family Journey to understand potential and consequences for relapse, in accordance with embodiments of the present disclosure.

FIG. 26 illustrates an information and access screen to encourage participants in a Family Journey to model healthy behaviors, in accordance with embodiments of the present disclosure.

FIG. 27 illustrates an information and access screen for participants in a Family Journey to understand aspects of continuing care following SUD treatment, in accordance with embodiments of the present disclosure.

FIG. 28 illustrates an information and access screen for participants in a Family Journey to understand treatment options and expectations after an inpatient stay, in accordance with embodiments of the present disclosure.

FIG. 29 illustrates an information and access screen to encourage participants in a Family Journey to stay connected to a support network, in accordance with embodiments of the present disclosure.

FIG. 30 illustrates an information and access screen to encourage participants in a Family Journey to consider mutual support groups to stave off isolation, in accordance with embodiments of the present disclosure.

FIG. 31 illustrates an information and access screen to encourage participants in a Family Journey to reflect on community built during the Family Journey, in accordance with embodiments of the present disclosure.

FIG. 32 illustrates an information and access screen for participants in a Family Journey to understand steps to rebuild trust in relationships impacted by SUD and recovery, in accordance with embodiments of the present disclosure.

FIG. 33 illustrates an information and access screen to congratulate participants in a Family Journey on acquiring new understanding and in aiding their loved one through treatment, in accordance with embodiments of the present disclosure.

FIG. 34 illustrates a surveys and forms home page on a mobile app, in accordance with embodiments of the present disclosure.

FIG. 35 illustrates a screen showing article and book content, in embodiments of the present disclosure.

FIG. 36 illustrates a screen of a mobile app showing links, podcasts, and video content, in embodiments of the present disclosure.

### DETAILED DESCRIPTION

Systems and methods for navigating a treatment process are provided. Particularly, systems and methods for assisting patient-adjacent people to navigate a treatment process are provided. More particularly, systems and methods for assisting patient-adjacent people to navigate a treatment process by contextualizing information and providing it at timely junctures in the treatment process are provided. The method may include associating a patient-adjacent person with a patient, assessing the treatment process that the patient is undergoing, and developing a patient-adjacent person program. The treatment process may include segments, junctures, and/or milestones. The program provides content to the patient-adjacent person and contextualizes the content based on at least one of the segments, junctures, and/or milestones.

The benefits of having an engaged and understanding patient-adjacent person include better treatment outcomes for the person in treatment and better quality of life for the adjacent person, who often serves in a caregiving or otherwise supporting role.

Treatment programs, for physical or mental health, are available in many different styles including, for example, in-patient and out-patient programs. Most treatment programs progress through defined steps and oftentimes the defined steps are at defined intervals. Health professional and/or caregivers are typically available to guide a patient through the steps and intervals. People adjacent to the patient (“patient-adjacent people”) such as family and friends, however, are commonly left to do their own research about what the patient is going through and what they, in turn, may experience.

The present disclosure offers a solution for addressing unmet concerns for patient-adjacent people in the behavioral health space. In some embodiments, the invention relates to a platform and system that anticipates stages, intervals, and/or junctures of a treatment program and patient reactions along at or within these stages, intervals, or junctures, and provides a patient-adjacent person program for guiding patient-adjacent people through those junctures. The system may set an initial configuration of the patient-adjacent person program and iteratively realign the program as the patient proceeds through treatment.

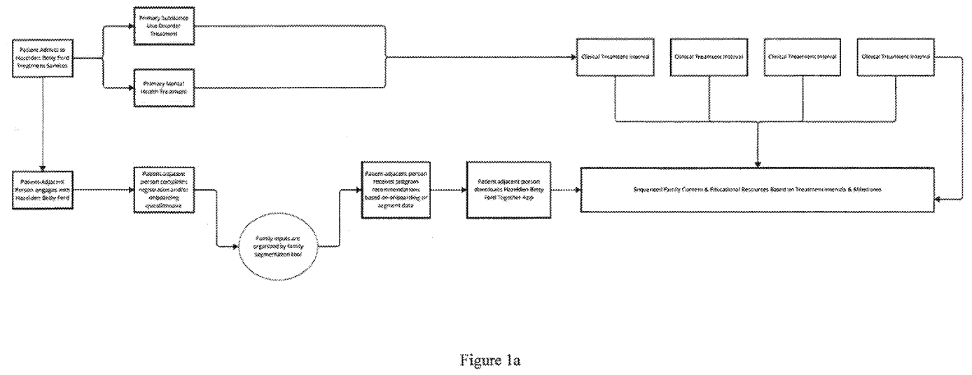

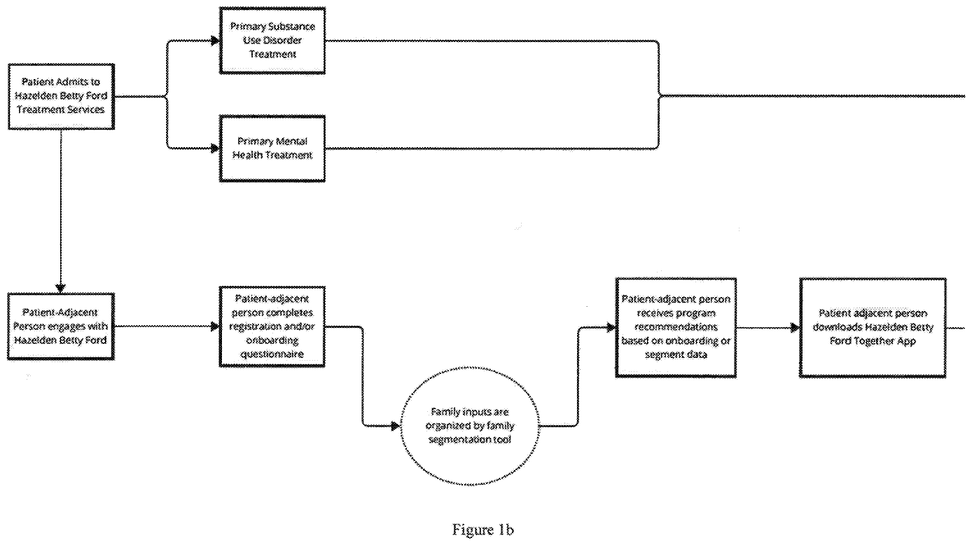

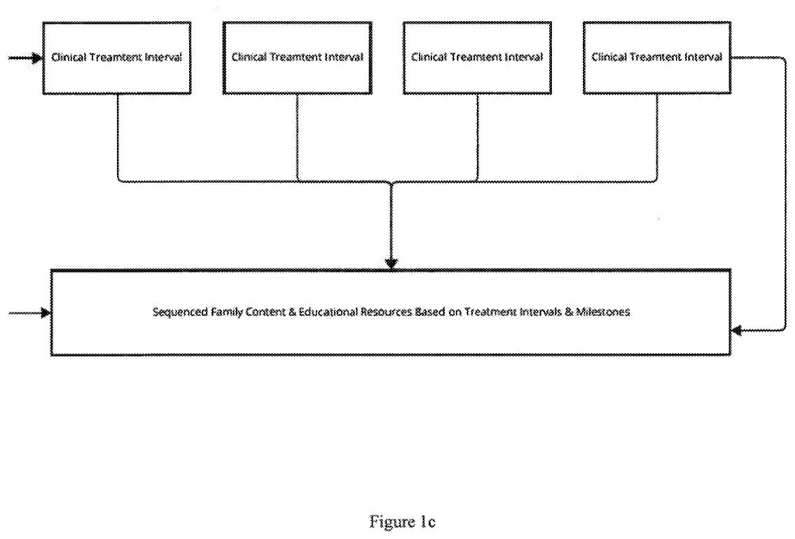

FIG. 1a illustrates a timeline showing patient navigation through a treatment process and patient-adjacent person navigation through patient-adjacent person program, in accordance with one embodiment. The upper half of FIG. 1a illustrates patient navigation through a treatment process. FIG. 1b illustrates admission and initial configuration set up of the timeline of FIG. 1a. FIG. 1c illustrates treatment and alignment of a patient-adjacent person program of the timeline of FIG. 1a.

Referring to patient navigation through the treatment process, a patient is admitted to a treatment facility. The patient goes through a series of stages and junctures associated with the treatment process. An early juncture is identification of what the patient is seeking treatment for. This may be, for example, for a SUD or for mental health treatment. Further, within each of those categories, there may be additional drill down aspects that impacts both the patient treatment program and the patient-adjacent person program. For example, the process, stages of the process, and junctures within the process may vary depending on whether a patient admitted for SUD treatment is seeking treatment for alcohol addiction versus opioid addiction. After admission and identification of treatment, the patient proceeds through a series of clinical treatment intervals.

Turning now to patient-adjacent person navigation through a treatment process, concurrent with or sometime after the patient is admitted to a treatment facility, the patient-adjacent person engages with the treatment facility.

The patient-adjacent person completes registration and/or onboarding questionnaire(s) and/or survey(s). The system and method may use information from a provider, the patient, and/or the patient-adjacent person to customize a patient-adjacent person program and to iteratively refine that program as the patient proceeds through the treatment process.

In some embodiments, the system and method may use one or more questionnaire(s) or survey(s) to gather information from the patient-adjacent person and/or the patient. These may be aimed at assessing the experience of patient-adjacent person and/or the patient with treatment programs, the likelihood of a good outcome, etc. These may be administered at any point during the treatment process. An initial survey and/or questionnaire may be used to develop an initial program for the patient-adjacent person. Subsequent surveys and/or questionnaires, as well as experience of both the patient and the patient-adjacent person during the treatment program, may be used to realign the patient-adjacent program as treatment progresses.

For example, as shown, a patient-adjacent person may complete a questionnaire or survey as part of onboarding. In some embodiments, the patient-adjacent person may be asked if they have had experience with treatment programs in the past-either as a patient, as a patient-adjacent person of the newly admitted patient, as a patient-adjacent person of a different patient, etc. Based on input given by the patient-adjacent person (and, in some cases, the patient) the patient-adjacent person's program is set in an initial configuration.

The system may provide the patient-adjacent person with content recommendations, resources, etc. based on the initial configuration of the patient-adjacent program. An app (referred to herein as the “Together App”) may be made available to the patient-adjacent person and may be set to the patient-adjacent person's initial program configuration. Based on the program configuration (whether the initial configuration or adjusted configurations), different resources and information may be pushed to the patient-adjacent person. These may further be customized based on a treatment juncture the patient is going through or input from the patient, a provider, or the patient-adjacent person. In some embodiments, the patient-adjacent person program may be static throughout the treatment process.

Referring to FIG. 1c, the patient proceeds through one or more clinical treatment intervals. Just before and after each of these treatment intervals may be considered a juncture. In some situations, one or more junctures may reside within a clinical treatment interval. Further, milestones may occur at or within a clinical treatment interval. Various content and educational resources may be pushed or otherwise made available to the patient-adjacent person based on a specific juncture milestone, or interval. During treatment, the patient-adjacent person program may be iteratively realigned based on experiences of the patient or the patient-adjacent person, information provided by the patient, the patient-adjacent person, or a provider, or other data.

System knowledge regarding timing of treatment intervals, junctures, and milestones may be preset based on a known treatment program or may correspond to data input by a provider. At a designated time (such as a treatment interval, juncture, or milestone), the system may reach out to a patient-adjacent person with content information useful for that time. The information may be generally useful information for the designated time or may be curated for the patient-adjacent person based on information provided by the patient, the patient-adjacent person, or a provider at or around the designated time. For example, the patient or the patient-adjacent person may be given a questionnaire or survey at or around the designated time and the answers given may influence the content or information provided to the patient-adjacent person.

In various embodiments, the system may be customized to address any suitable population. In some embodiments, the user may be a patient-adjacent person. More specifically, the user may be a friend of the patient, a family member of the patient, a coworker of the patient, or other person who is not the patient. In other embodiments, the user may be a patient.

The system and method may be customized to any treatment program having intervals, junctures, or milestones therein. It is to be appreciated that intervals, junctures, and milestones are used generally interchangeably herein as being times at which the system may interact with a patient-adjacent person or provide content, information, or resources to a patient-adjacent person. Use of a specific term is not intended to be limiting. The system and method may be customized to an addiction treatment program-such as a program for treatment of alcohol or drug addiction. The system may be configured to be fully automated or to be responsive to input from a service provider and/or from a user.

There is a large volume of information available to patients of a treatment program and to adjacent people of patients of a treatment program. There are books, blogs, informative websites, and the like. A problem is that there is nothing that contextualizes that information for a user. More specifically, there is nothing that contextualizes the information in the right time frame to maximize applicability of the information. For example, the issues that may arise at one interval, juncture, or milestone are not the issues that may arise at another interval, juncture, or milestone. Thus, a patient or patient-adjacent person must sort through whatever information they have and find the information specific to that juncture in advance or, or concurrent with, the juncture. Finding and identifying the information after the juncture has passed decreases the usefulness of the information.

The system may be designed to address just-in-time variables associated with an interval, juncture, or milestone. For example, the system may reach out to a patient-adjacent person to say “PATIENT met with a group today to discuss THIS. You may want to expect a call from PATIENT and that is very normal.” The specific content may be based on timing but also on data about the patient and/or the patient-adjacent person. For example, if the patient is progressing positively through the treatment process, the content may be of a first nature. If the patient is exhibiting negative behavior through the treatment process, the content may be of a second nature.

Timing of communication by the system for an interval, juncture, or milestone may be based on a preset schedule. For example, a program may be designed to last for 10 weeks with known junctures at 2, 4, 6, and 8 weeks. The system may be configured to automatically communicate with the patient-adjacent person at 2, 4, 6, and 8 weeks. Alternatively, timing of communication by the system for an interval, juncture, or milestone may be based on service provider input that an interval, juncture, or milestone is approaching or has occurred.

The patient's progress through the treatment program may affect the both the delivery timing and the content delivered. For instance, a patient may need more assistance with reaching a specific milestone, segment, or juncture than another. Content delivered to the patient-adjacent person at or near this segment, milestone, or juncture, may be made more intensive or supportive. Likewise, if a patient is progressing rapidly or leaves the program early, the systems and methods of the present disclosure can accelerate delivery to correspond to the milestone, segment, or juncture actually experienced by the patient. The data and information relating to patient progress may be entered by the provider, or accessed from electronic health record available in a data repository.

The system may communicate with users in any suitable manner now or later developed. For example, the system may be an app, the system may be accessible via a web portal, the system may use a texting relay, or other technology now or later developed. The system may have 1-way communication, may have 2-way communication, may alternate between 1-way and 2-way communication based on the interval, juncture, or milestone. More specifically, the system may provide preset information at a juncture, may communicate with a user to provide one or more of a plurality of information at a juncture, and/or may provide information to a user upon request by the user.

An example system for delivering contextualized content may include a server that may include component modules, processors, services, and applications to sort, transform, and deliver contextualized content. The server may include any number of processing devices that include various modules from server. Each module may include either a software or hardware solution, such as a computer processor, that meets the needs of the system. The server may include or have access to a data collection processor to collect, retrieve, or access data from one or more data sources, and a data repository. Server may be in communication with client devices over network (e.g., the Internet or cellular communication) through communication processor.

Client device can be any electronic device with an input device and graphical user interface (GUI), such as computer, personal computer, wearable computer, server, personal digital assistant (“PDA”), hybrid PDA/mobile phone, mobile phone, electronic book reader, set-top box, camera, appliance (e.g., a thermostat or refrigerator), controller, digital media player, watch, eyewear, a home or car device, Internet of Things (“IoT”) devices, virtual reality or augmented reality devices, and the like. In some embodiments of system, client device runs a browser or app that provides a user interface for interacting with content (such as web pages, video content, and/or the like) from server. Transmission of content and/or data in response to requests from client device can take place using any known protocols and languages.

In some exemplary embodiments, the care provider can interact with the server to provide or release contextualized content to one or more client devices for maintaining the software, logic, components, settings, etc. necessary for storing and executing the methods described herein, creating a host device. In this manner, the league administrator use the host device in his or her control to make contextualized content available to each of the plurality of participant client device prior to and during a treatment program.

The system can perform specific operations to create and deliver contextualized content, such as various manners of processing input data into training data (e.g., tokenizing input data; forming input data into prompts and/or completions), and managing training and other machine learning model configuration processes. Various components of the system can be implemented using one or more computer systems, which may be provided on the same or different processors (e.g., processors communicatively coupled via wired and/or wireless connections).

The server may be configured to retrieve and store content, resources, forms, and templates within data repository. For example, the data repository can include one or more databases to store, for example, clinical content, links, videos, and other program resources (that may be generated by various machine learning models and/or algorithms), along with any other information or data arrangements relied upon by the methods disclosed herein. Certain databases of the data repository may be made accessible to client devices.

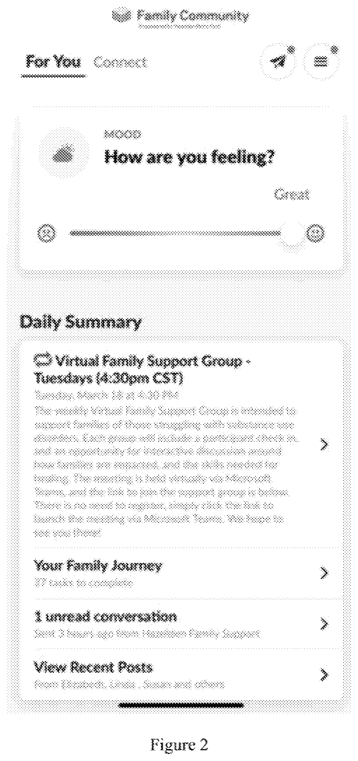

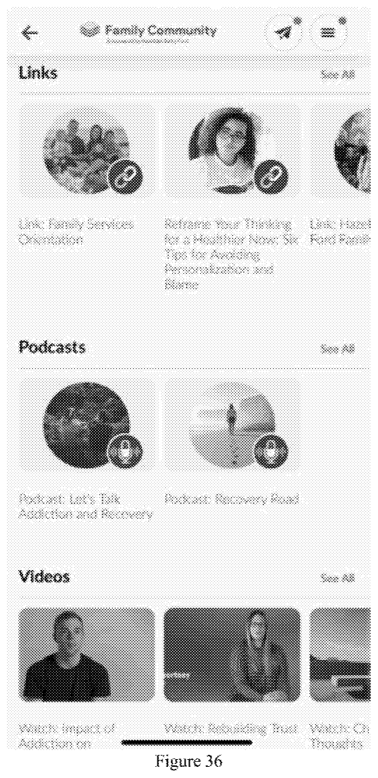

FIGS. 2-33 and 34-36 illustrate screenshots of aspects of an app for a patient-adjacent person navigating a treatment process. In general, in some embodiments, the app may include a Feed feature, a Wellness feature, a Care Journeys feature, a Messaging feature, an Explore feature, and a Profile. Some aspects of these will be discussed and/or illustrated below.

The Feed feature allows a patient-adjacent person to view posts from a provider, from other users of the app, or from other enabled users. Patient-adjacent users may view proprietary content, updates, and posts.

The Wellness feature allows a patient-adjacent person to keep a gratitude journal and track their mood.

The Care Journeys feature offers content that can support patient-adjacent people on their journey of healing with the patient. Various topical resources may be sequenced in order, aligning with the arc of clinical treatment.

The Messaging feature allows patient-adjacent people to contact/message the treatment facility to find resources and guidance about programs and resources.

The Explore feature allows patient-adjacent people to view resources such as articles, videos, and books that can assist with navigating the treatment process. These can be viewed on demand in the order the user chooses.

The Profile feature allows a patient-adjacent person to maintain a profile, updating milestones, adjusting notifications, and managing settings. Updates to milestones may be used to adjust a patient-adjacent person program.

FIG. 2 illustrates a daily welcome page, in accordance with one embodiment. The welcome page can provide information about content or resources available and can also assess status of the patient-adjacent person. For example, the welcome page can ask the patient-adjacent person to rate their mood and this rating may be used by the system to further cultivate information and content, as well as a prescribed patient-adjacent program.

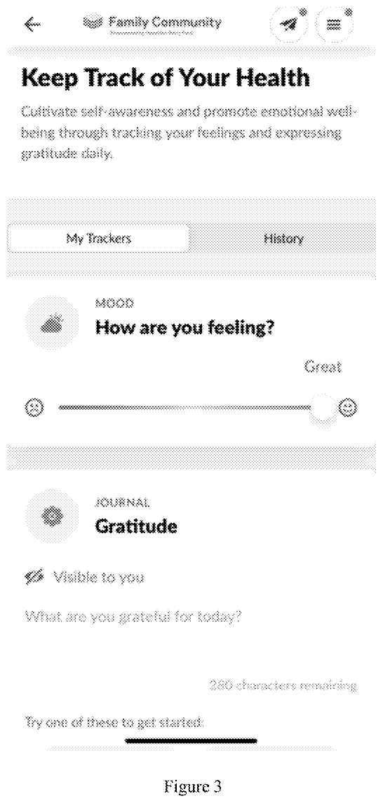

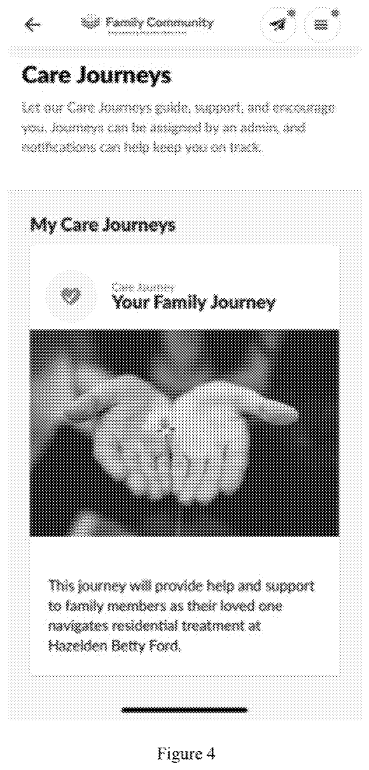

FIG. 3 illustrates a self-tracker page, in accordance with one embodiment. This page may be used for patient-adjacent person to rate their mood or to input information such as experiences, what they are grateful for, etc. In some embodiments, the app may be configured with a keyword identifier to identify words input by the patient-adjacent person to further cultivate information and content, as well as a prescribed patient-adjacent program.

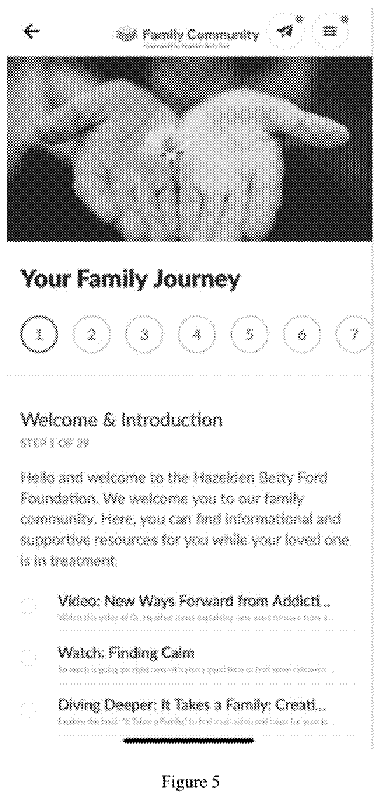

The app may provide one or more journeys for a patient-adjacent person to select. These journeys are sequenced patient-adjacent programs that deliver content and recommendations relating or corresponding to stage, junctures, or milestones in the patient journey. FIGS. 4-33 illustrate aspects of a Family Journey, in accordance with one embodiment. The Family Journey is designed to help and support family members of a patient as the patient navigates a treatment process, delivering or otherwise making available customized content at an interval, juncture, or milestone.

FIG. 4 illustrates a cover page of the Family Journey. FIGS. 5-33 illustrate specific informational and access screens of the Journey. Each screen may be designed to correspond with a segments, junctures, or milestones of a patient treatment process and may include content, information, or links (i.e., resources and materials) related to that segment, juncture, or milestone. Each of the screen corresponds with a step of the Journey (Steps 1 through 29 in the embodiment shown). The screens may be static or may vary based on the experience of the patient or the patient-adjacent person. Further, the screens may be all available at once or may be released upon approaching, reaching, or passing a segment, juncture, or milestone. “Screen” or “screens” refer to the presentation of content as delivered to the GUI of a client device accessible to a patient-adjacent persons

The timing of screen release described below for FIGS. 5-33 is accordingly for example only, and does not necessarily fix the delivery or presentation of content to a given timeline. In presently preferred embodiments, the screens are delivered in the sequence presented in FIGS. 5-33, which corresponds to delivery at or before relevant junctures in the treatment process. References to a family member are applicable to any patient-adjacent person.

FIG. 5 illustrates a welcome screen. The welcome screen may be presented to the family member upon a patient checking into to a treatment program. The welcome screen may provide or link to, for example, introductory content relating to a facility or care provider, as well as content encouraging compassion for the person in treatment and calm for the family member.

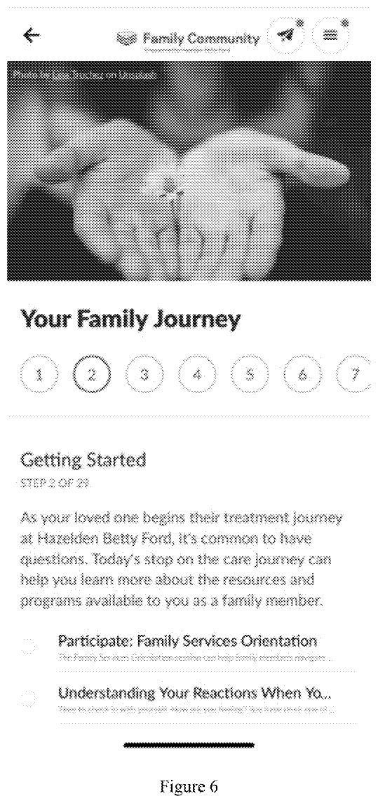

FIG. 6 illustrates a screen for orientation and onboarding. The orientation and onboarding screen may be presented to the family member in days following welcome and patient check-in. The orientation and onboarding screen may provide or link to, for example, resources to aid navigating the treatment process(es) and resources available through the care provider, as well as content encouraging the family member to address and reflect on treatment related emotions.

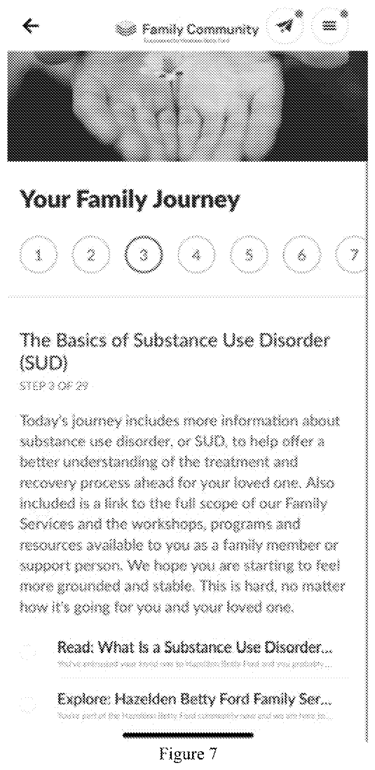

FIG. 7 illustrates a screen for education on SUD, or other treatment specific health issues. The behavioral health education screen may be presented to the family member in days following orientation and onboarding. The education screen may provide or link to, for example, resources for gaining understanding of the maladies affecting the person in treatment, as well as content encouraging the family member to explore support programs of the care provider.

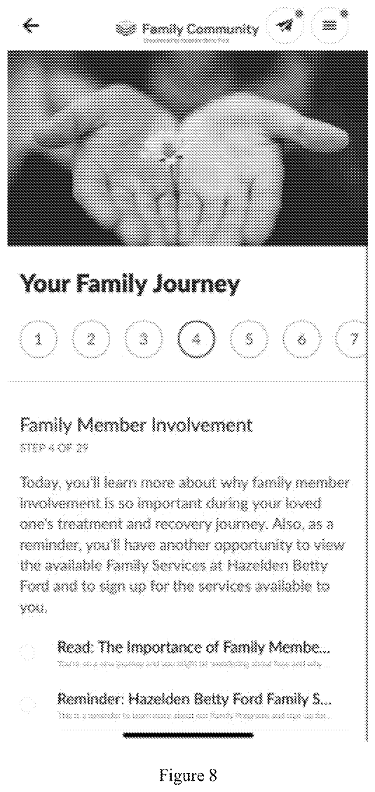

FIG. 8 illustrates a screen for highlighting the importance of family member involvement in the treatment. The involvement screen may be presented to the family member in days following SUD education (e.g., day 3 or step 4 of the program). The education screen may provide or link to, for example, resources for gaining an understanding how to stay involved in the treatment process, as well as content encouraging the family member to explore support programs of the care provider and other resources previously presented through the app.

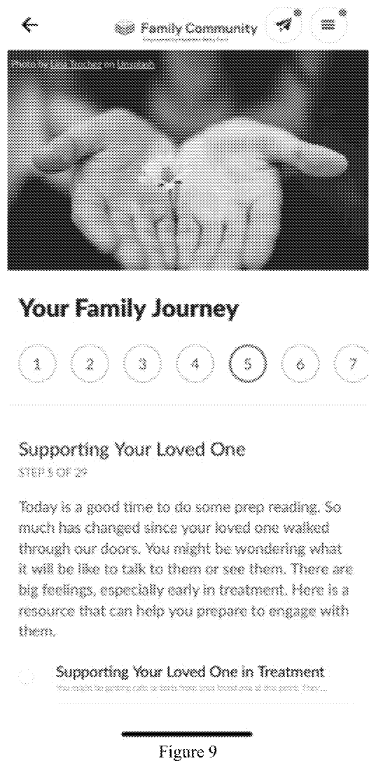

FIG. 9 illustrates a screen for offering tips and resources for providing direct support to the person in treatment. The direct support screen may be presented to the family member in days following involvement (e.g., day 4 or step 5 of the program). The education screen may provide or link to, for example, resources for helping the family member answer direct questions or concerns from the person in treatment, as well as content encouraging the family member to prepare for future communications and struggles.

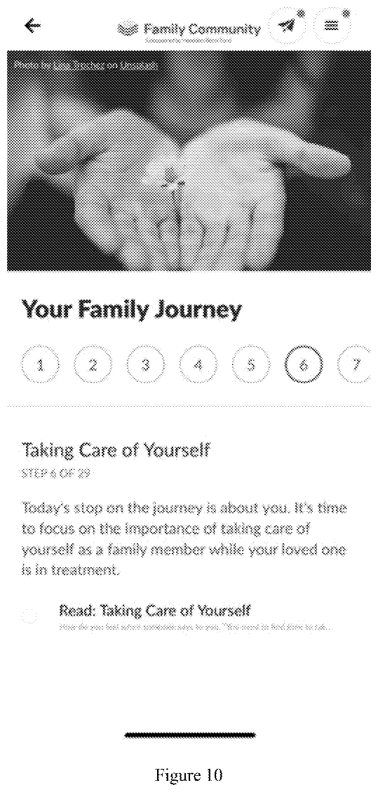

FIG. 10 illustrates a screen for educating the family member on self-care. The self-care education screen may be presented to the family member in days following direct support (e.g., day 5 or step 6 of the program). The self-care screen may provide or link to, for example, resources for addressing and taking care of the family member's own health while otherwise supporting the person in treatment, as well as techniques to address one's own journey.

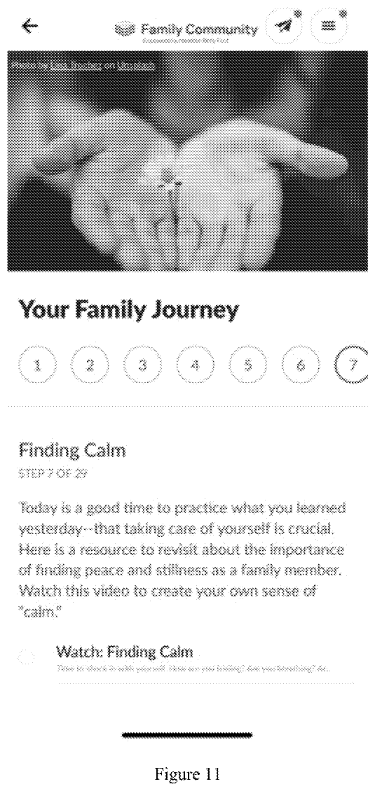

FIG. 11 illustrates a screen for putting self-care techniques into practice and encouraging the family member to find a state of calm. The calm screen may be presented to the family member in days following the self-care education screen (e.g., day 6 or step 7 of the program). The calm screen may provide or link to, for example, resources for creating a sense of calm, as well as content encouraging the family member to assess their own mental state.

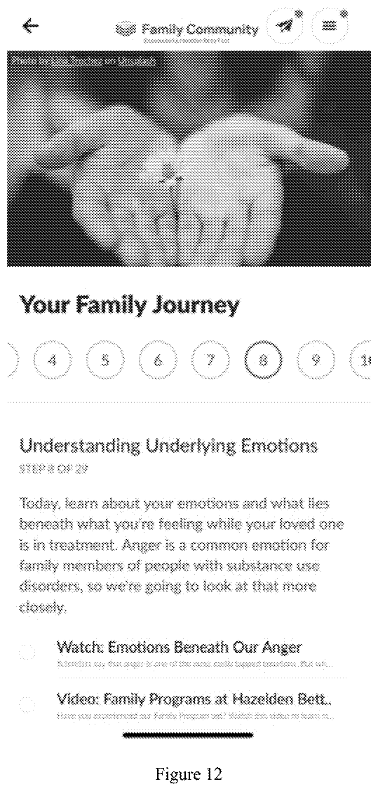

FIG. 12 illustrates a screen for encouraging the family member to understand the emotions (e.g., anger) that the family member may be experiencing. The emotional reckoning screen may be presented to the family member in days following the self-care technique screen (e.g., day 7 or step 8 of the program). The emotional reckoning screen may provide or link to, for example, resources for interrogating underlying reasons for emotions, as well as content encouraging the family member to explore support programs of the care provider and other resources previously presented through the app.

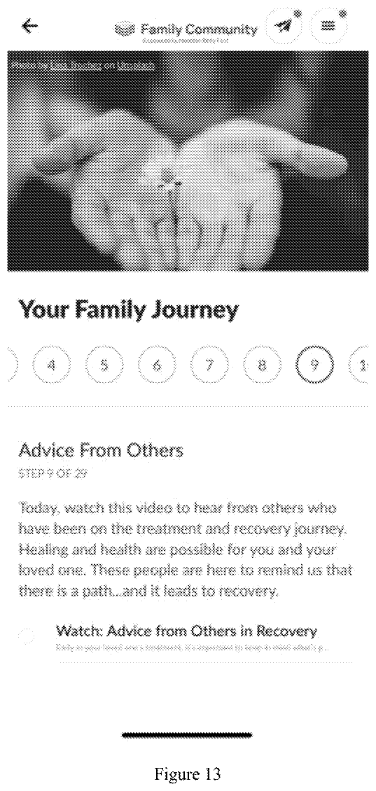

FIG. 13 illustrates a screen offering testimony from others who have experienced the treatment process as the patient and/or a patient-adjacent person. The shared experience screen may be presented to the family member in days following the emotional reckoning screen (e.g., day 8 or step 9 of the program). The shared experience screen may provide or link to, for example, resources relating to stories and testimonies of individuals in recovery.

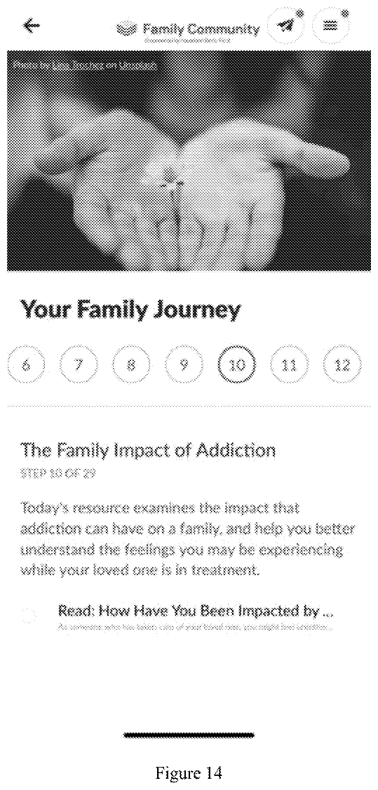

FIG. 14 illustrates a screen for evaluating the impact of the treatment process on the family member. The family impact screen may be presented to the family member in days following the shared experience screen (e.g., day 9 or step 10 of the program). The family impact screen may provide or link to, for example, resources for assessing and cataloging the extent of impact on the family member.

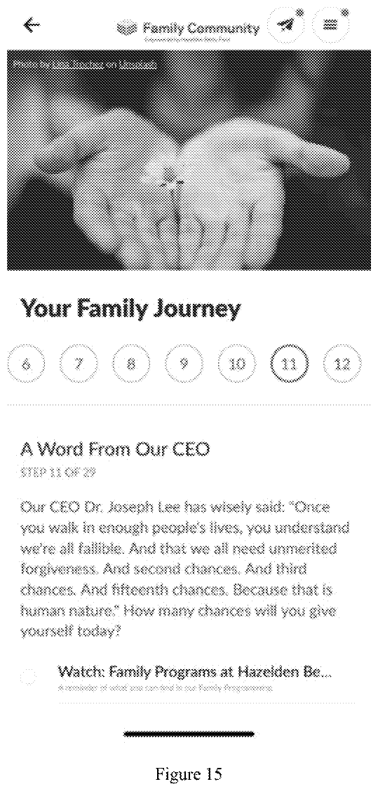

FIG. 15 illustrates a screen for offering a message of support from a leader or care giver. The support message may be presented to the family member in days following the family impact screen (e.g., day 10 or step 11 of the program). The support may provide or link to, for example, a video thanking the family member for his or her support and encouraging the family member to continue on the Family Journey.

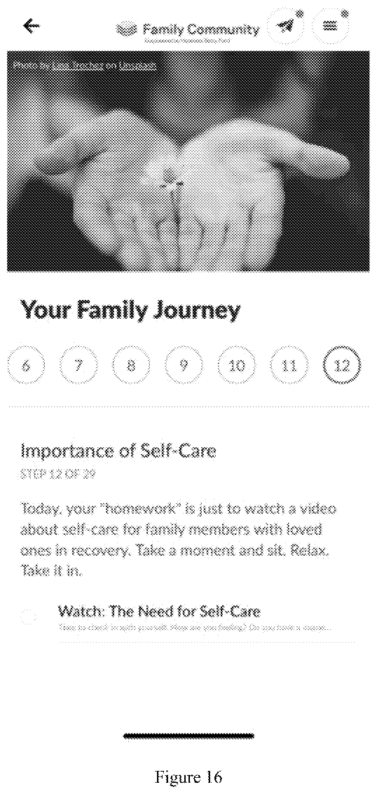

FIG. 16 illustrates a screen for putting self-care techniques into practice and encouraging the family member to find a state of relaxation. The calm screen may be presented to the family member in days following the message of support (e.g., day 11 or step 12 of the program). The self-care screen may provide or link to, for example, resources for creating a sense of calm, as well as content encouraging the family member to assess their own mental state.

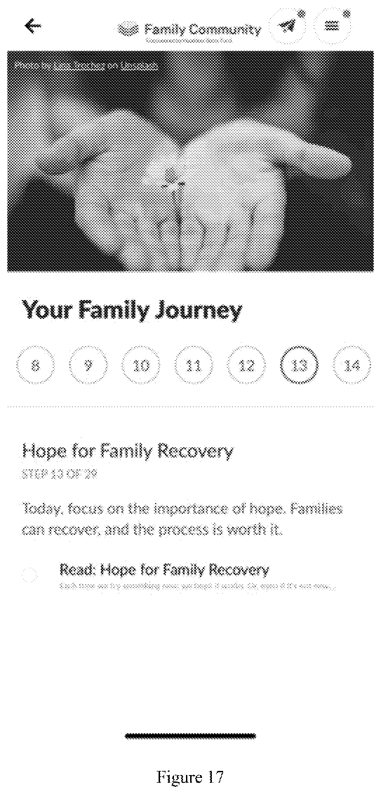

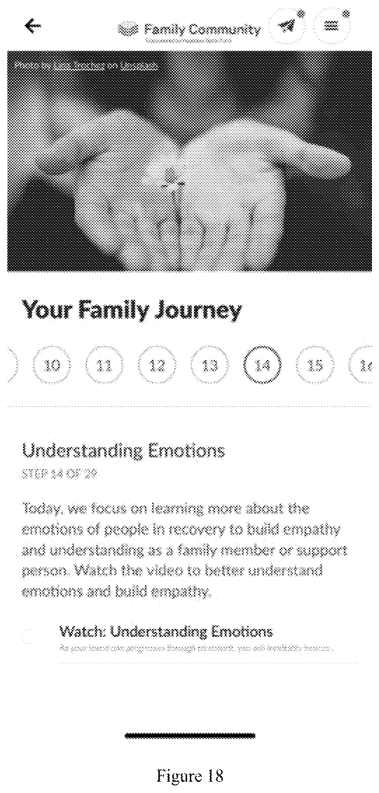

FIG. 17 illustrates a screen for encouraging the family member to hold out hope in the treatment process and promise of recovery for the person in treatment. The hope screen may be presented to the family member in days following a self-care education screen (e.g., day 12 or step 13 of the program). The hope screen may provide or link to, for example, resources for curating a sense of hope for the person in treatment and the family.

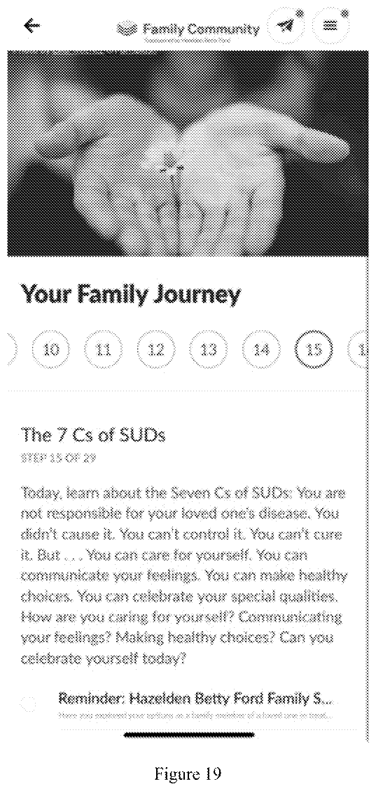

FIG. 18 illustrates a screen for encouraging the family member to understand the emotions associated with recovery following the treatment process. The empathy screen may be presented to the family member in days following the hope screen (e.g., day 13 or step 14 of the program). The empathy screen may provide or link to, for example, resources for revealing and understanding emotions expressed by the person in treatment, as well as testimonials.

FIG. 19 illustrates a screen for helping family member contextualize SUD (or underlying reason for treatment) using the 7 Cs (cause, control, cure, care, communicate, choice, and celebrate). The contextualization screen may be presented to the family member in days following the empathy education screen (e.g., day 14 or step 15 of the program). The contextualization screen may provide or link to, for example, resources for encouraging the family member, as well as content encouraging the family member to explore support programs of the care provider and other resources previously presented through the app.

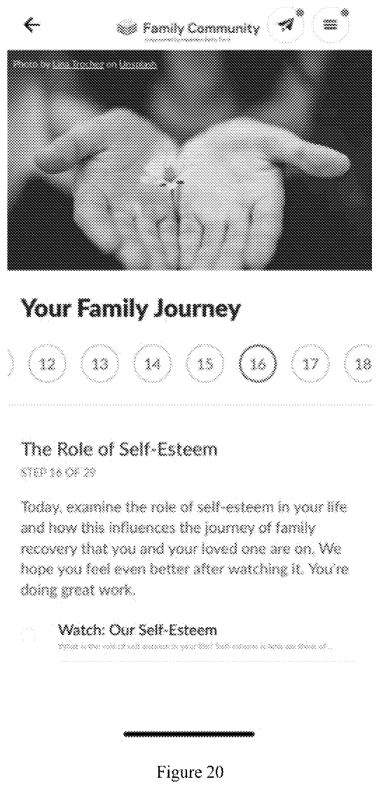

FIG. 20 illustrates a screen for encouraging the family member to examine self-esteem and its influence on both treatment and recovery. The self-esteem screen may be presented to the family member in days following the contextualization screen (e.g., day 15or step 16 of the program). The calm screen may provide or link to, for example, resources for creating a sense of calm, as well as content encouraging the family member to assess their own mental state.

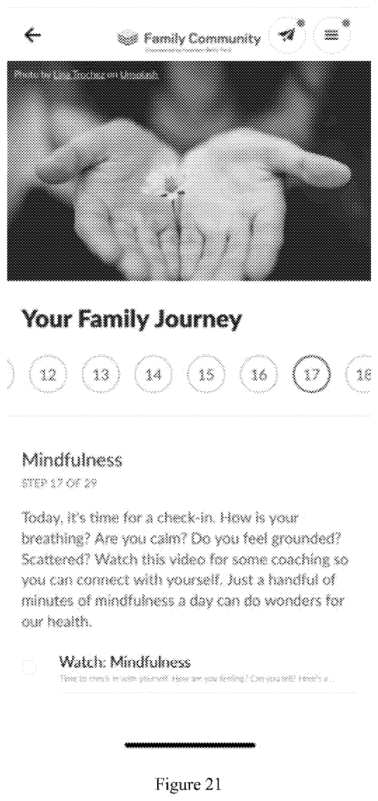

FIG. 21 illustrates a screen for putting self-care techniques into practice and encouraging the family member to find a state of mindfulness. The mindfulness screen may be presented to the family member in days following the self-esteem screen (e.g., day 16 or step 17 of the program). The self-care screen may provide or link to, for example, resources for creating a sense of calm, as well as content encouraging the family member to assess their own mental state.

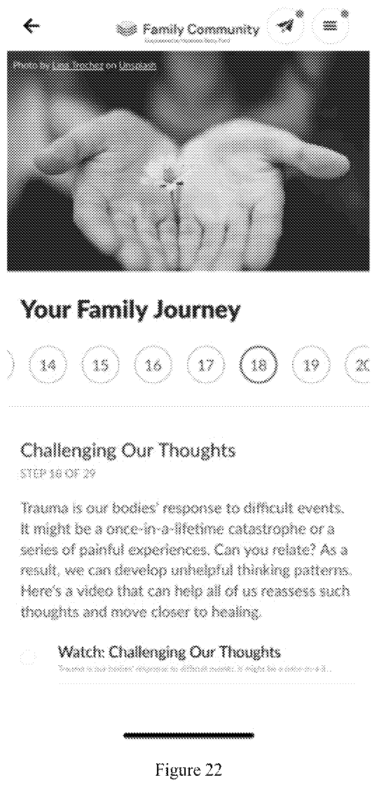

FIG. 22 illustrates a screen for encouraging the family member to interrogate unhelpful thought patterns. The thought challenge screen may be presented to the family member in days following a self-care education screen (e.g., day 17 or step 18 of the program). The thought challenge screen may provide or link to, for example, resources for aiding the family member is reassessing difficult or unhelpful thoughts stemming from trauma, and moving towards healing.

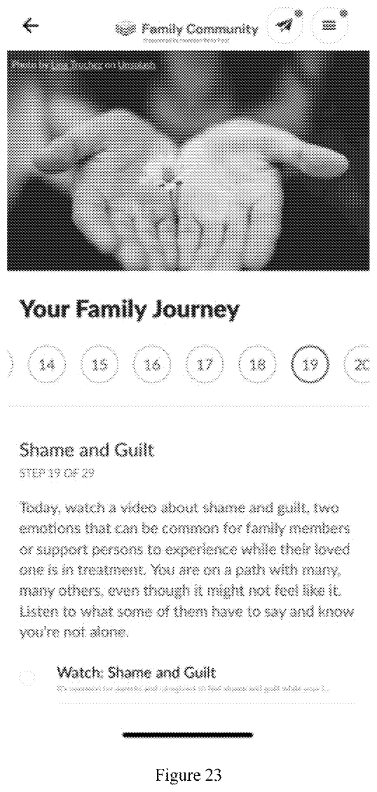

FIG. 23 illustrates a screen for encouraging the family member to understand the shame and guilt that the family member may be experiencing. This emotional reckoning screen may be presented to the family member in days following the thought challenge screen (e.g., day 18 or step 19 of the program). The thought challenge screen may provide or link to, for example, resources for managing shame and guilt, along with addressing stigma.

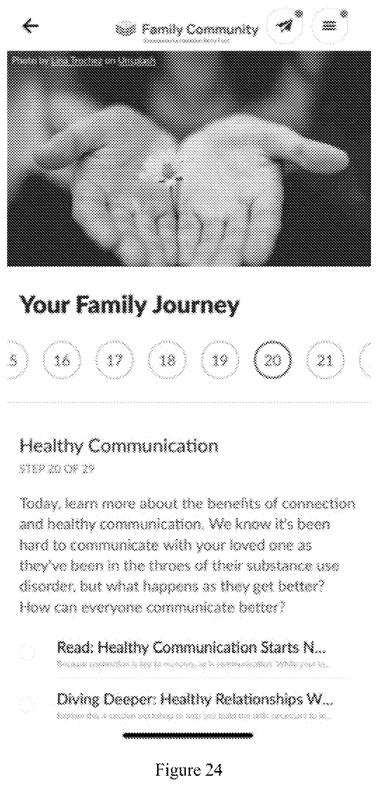

FIG. 24 illustrates a screen for encouraging healthy communication amongst the family member, person in treatment, and the care giver. This healthy communication screen may be presented to the family member in days following the an emotional reckoning screen (e.g., day 19 or step 20 of the program). The healthy communication screen may provide or link to, for example, resources and workshops for establishing effective communication techniques.

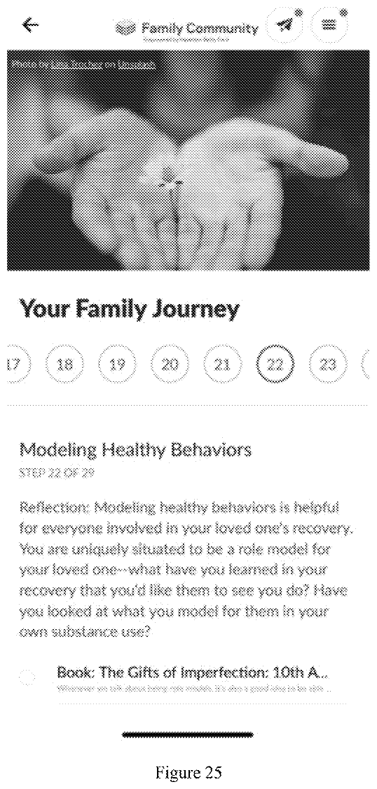

FIG. 25 illustrates a screen for educating the family member on relapse. The relapse education screen may be presented to the family member some days after the healthy communication screen (e.g., day 20 or step 21 of the program), when then program is nearing completion. The relapse screen may provide or link to, for example, resources to help the family member prepare for a relapse, as well as content designed to aid the family member in supporting a person in relapse.

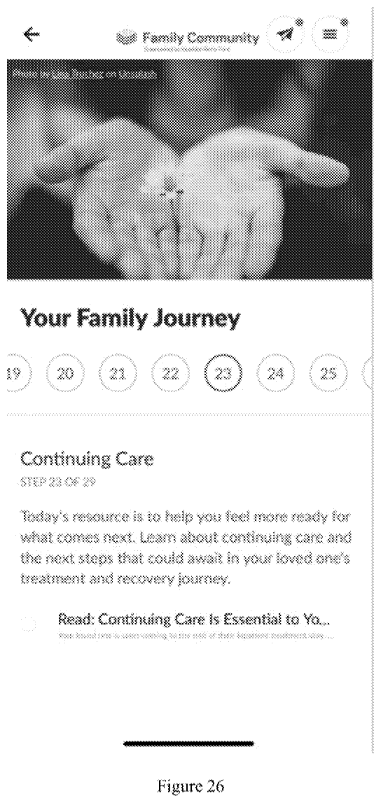

FIG. 26 illustrates a screen for encouraging the family member to model health behaviors and act as a role model to aid the person in treatment. This role model screen may be presented to the family member in days following the relapse education screen (e.g., day 21 or step 22 of the program). The role model screen may provide or link to, for example, resources and workshops for establishing effective communication techniques.

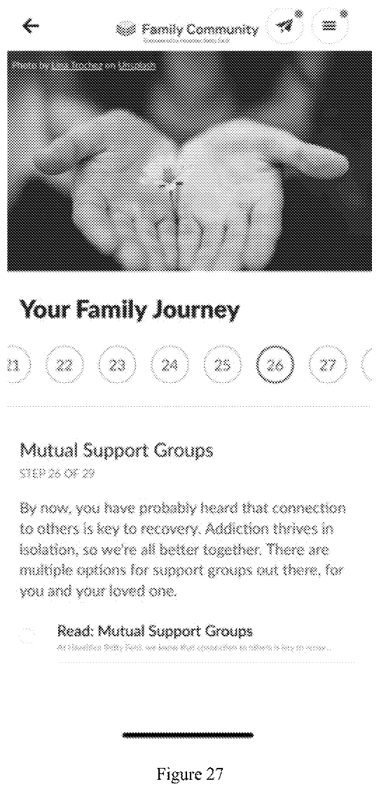

FIG. 27 illustrates a screen for educating the family member about continuing care following the treatment process. The continuing care education screen may be presented to the family member after the role modeling screen (e.g., day 22 or step 23 of the program), when then program is nearing completion. The continuing care education screen may provide or link to, for example, resources to help the family member prepare for the end of treatment, as well as content encouraging the family member to explore support programs of the care provider and other resources previously presented through the app.

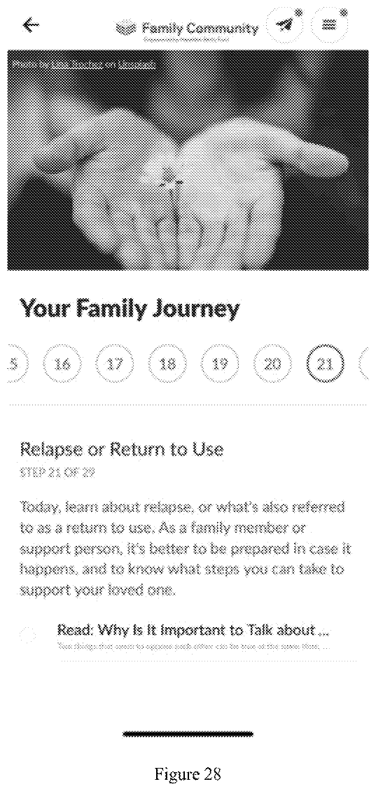

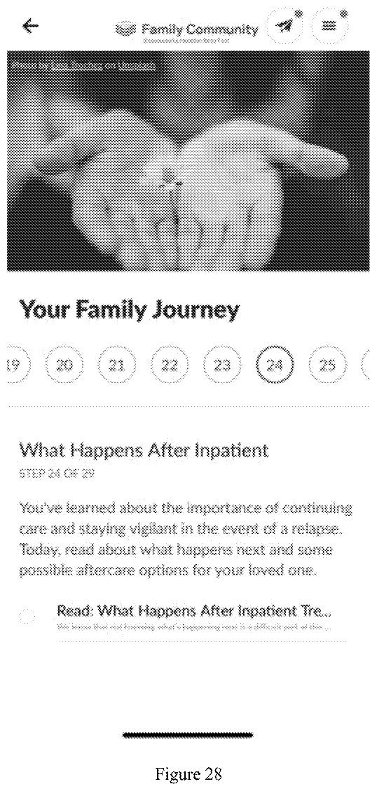

FIG. 28 illustrates a screen for educating the family member about the availability of mutual support groups for both the family member and the person in treatment. The support group screen may be presented to the family member after the role continuing care screen (e.g., day 23 or step 24 of the program). The support group screen may provide or link to, for example, resources to help the connect the family members with groups supporting post-care recovery, and encouraging the avoidance of isolation.

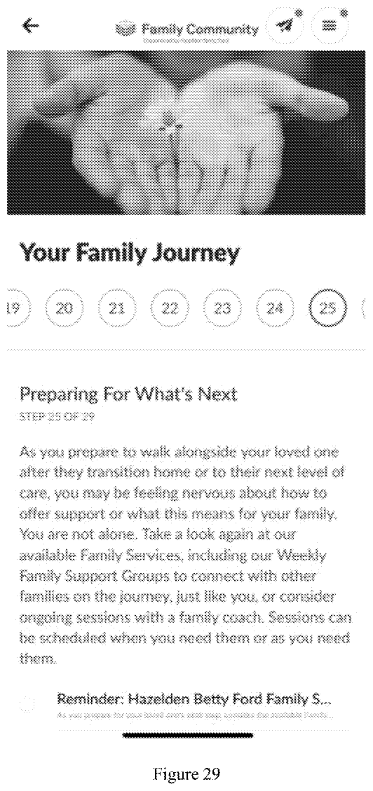

FIG. 29 illustrates a screen for educating the family member about discharge following the treatment process. The discharge education screen may be presented to the family member in days following the mutual support group screen (e.g., day 24 or step 25 of the program), when then program is nearing completion. The discharge screen may provide or link to, for example, resources to help the family member prepare for the end of treatment, as well as material encouraging the family member to explore other after care options for the person in treatment.

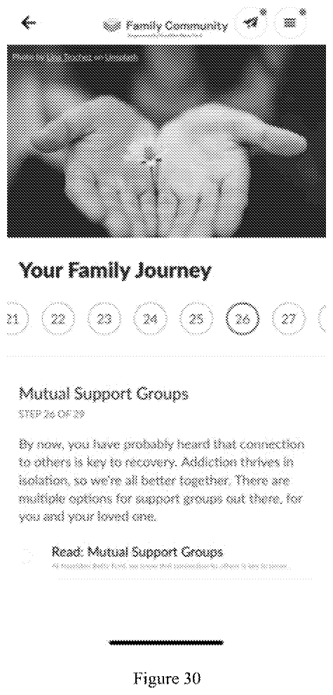

FIG. 30 illustrates a screen for educating the family member about supporting the person in treatment following discharge. The post-discharge support screen may be presented to the family member after the discharge education screen (e.g., day 25 or step 26 of the program), when then program is nearing completion. The post-discharge support screen may provide or link to, for example, resources to help the family member prepare for the end of treatment, as well as content encouraging the family member to explore support group and other service options for the person in treatment.

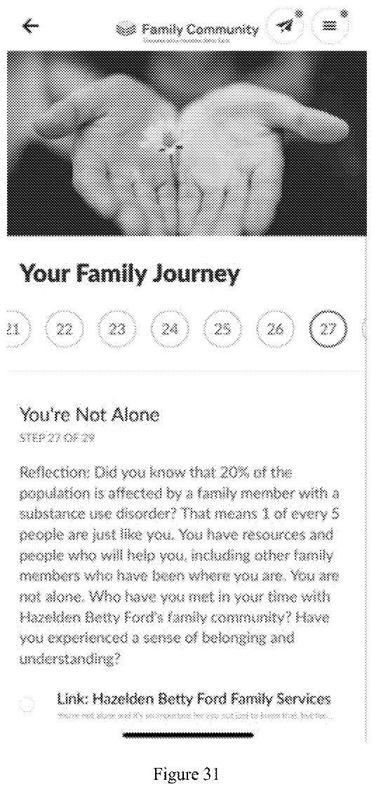

FIG. 31 illustrates a screen for encouraging the family member to reflect on the treatment process and the connections made. The reflection opportunity screen may be presented to the family member in days after the end of treatment screen (e.g., day 26 or step 27 of the program), when then program is nearing completion.

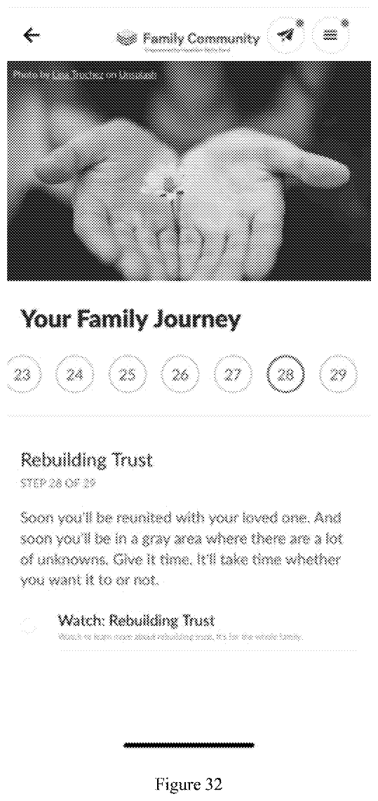

FIG. 32 illustrates a screen illustrates a screen for helping the family member rebuild trust with the person in treatment. This trust building screen may be presented to the family member in days following the self-reflection screen (e.g., day 27 or step 28 of the program). The trust screen may provide or link to, for example, resources and workshops for re-establishing trust amongst family members.

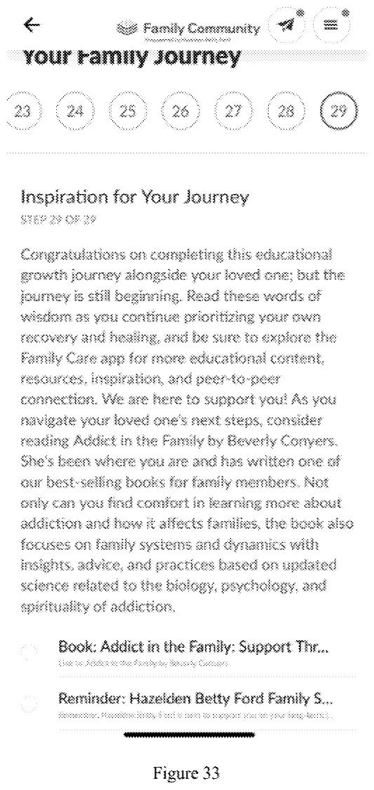

FIG. 33 illustrates a congratulatory screen, delivered on the completion of the treatment program (e.g., day 28 or step 29 of the program).

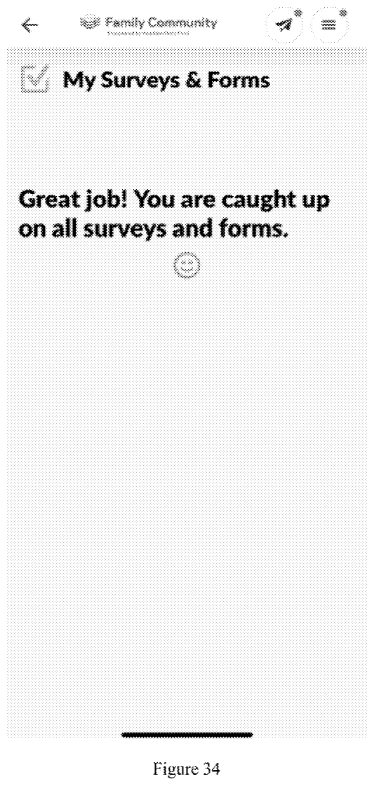

The system may use surveys, questionnaires, and forms to gather information from a patient-adjacent person. These surveys, questionnaires, and forms may solicit information that can be used by the system to develop or realign a patient-adjacent program. FIG. 34 illustrates a surveys and forms home page on the app, in accordance with embodiments of the present disclosure.

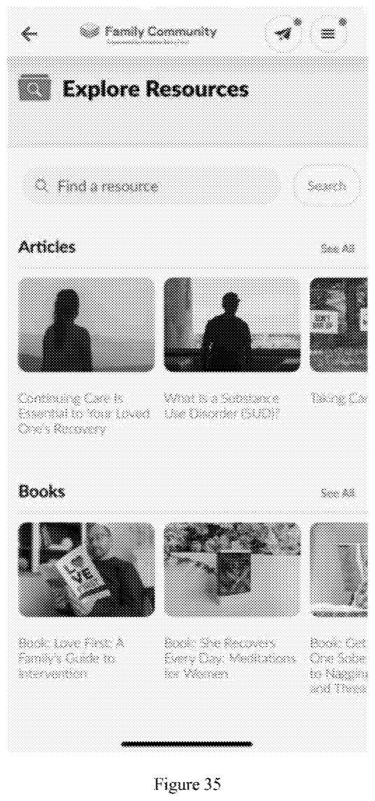

In addition to content associated with specific segments, junctures, or milestones and, in some embodiments curated to a specific patient or patient-adjacent user experience, the system and method may provide more generalized content. FIG. 35 illustrates a page of an app showing article and book content, in one embodiment. FIG. 376 illustrates a page of an app showing links, podcasts, and video content, in one embodiment.

The system may and method may offer resources designed to integrate a patient-adjacent person into the care journey as experienced by the patient. For instance, a clinical care journey may include a care sequence for the patient, where each step in the care sequence is supported by specific interventions, explorations, discussions, or expectations. The systems and methods may present resources to the patient-adjacent person that align with a step in the care sequence currently experienced by the patient. The resources may identify and offer a clinical explanation for a care sequence step, explain its relevance to the treatment program and the patient, offer guidance on how the patient-adjacent person may respond to and support the patient, as well as offer support for well-being of the patient-adjacent person. A consistent structure reinforces the navigation method by translating patient treatment milestones into family-appropriate interpretation and guidance. The care sequence resources may be delivered to the patient-adjacent person in specific series, or may be made available to for access the direction and discretion of the patient-adjacent person.

The system for navigating a patient-adjacent user through a treatment process may use machine learning to enhance user experience and identify junctures wherein it may be useful to interact with a user and/or identify information that may be useful to provide to a user at a given juncture. Machine learning is a form of artificial intelligence (AI) that teaches computers to learn and improve upon past experiences. It works by exploring data and identifying patterns. The processor is programmed with an initial data-defined pattern or set of rules and that pattern is automated with machine learning. The machine learning model is trained using users'interactions with the system (click throughs, requests for more information, etc.).

If desired, the machine readable instructions can include a machine learning algorithm, for example, to improve interactive functions with the user. The system may be configured to use free AI models developed by Microsoft, OpenAI, Anthropic, or other machine learning or machine learning algorithms. The processor thus may include an algorithm for identifying junctures wherein it would be useful to interact with a user and a training set for facilitating machine learning for identifying junctures wherein it would be useful to interact with a user. The training set may include data relating to a treatment programs, clinical understanding, and user engagement.

Machine learning systems, including but not limited to LLMs, can be used to generate text data, images, and data of other modalities in a more responsive manner to real-time conditions, including generating strings of text data that may not be provided in the same manner in existing sources, yet may still meet criteria for useful text information, such as relevance, style, and coherence. For example, LLMs can predict text data based at least on inputted prompts and by being configured (e.g., trained, modified, updated, fine-tuned) according to training data representative of the text data to predict or otherwise generate. Systems and methods in accordance with the present disclosure can use machine learning models, including LLMs and other generative artificial systems, to capture data, including but not limited to unstructured knowledge from various data sources, and process the data to accurately generate outputs, including in structured data formats for various purposes.

In various implementations, the systems can include a plurality of AI models that may be configured using integrated or disparate data sources. This can facilitate more integrated user experiences or more specialized (and/or lower computational usage for) data processing and output generation. Outputs from one or more first systems, such as one or more first algorithms or AI models, can be provided at least as part of inputs to one or more second systems, such as one or more second algorithms or AI models. For example, a first language model can be configured to process unstructured inputs (e.g., text, speech, images, etc.) into a structure output format compatible for view or use by a second system, and eventual presentation to on the display screen of client device. AI models can include, for example and without limitation, one or more language models, LLMs, attention-based neural networks, transformer-based neural networks, generative pretrained transformer (GPT) models, bidirectional encoder representations from transformers (BERT) models, encoder/decoder models, sequence to sequence models, autoencoder models, generative adversarial networks (GANs), convolutional neural networks (CNNs), recurrent neural networks (RNNs), diffusion models (e.g., denoising diffusion probabilistic models (DDPMs)), or various combinations thereof.

One embodiment comprises a system and method for guiding a user through a behavioral health program. With specific reference to a behavioral health embodiment, it is common that a patient goes through a relatively predictable treatment process. The system and method provide people adjacent to the patient with information at defined junctures. This information may include guidance on what behavior may be normal or abnormal from a patient at what junctures in a treatment process. More specifically, the system and method may provide step by step guidance tailored to what a patient is going through at a juncture and what the patient-adjacent people can expect.

The system and method may further provide a mechanism by which a user, such as a patient-adjacent person, can input their experience at or before a juncture such that the information provided may be tailored to that user. For example, using the system, the patient-adjacent person can answer questions or prompts and can be given advice based on the answers given.

In use, the patient-adjacent person sets up an account on the system, for example at patient drop off, after a first call to an intake center expressing concern for the patient, etc. The system may prompt the user to give details such as what treatment the patient is seeking, what treatment the patient has previously undergone, and what experience the patient-adjacent person has with the treatment or what the patient is being treated for.

The system may be configured to provide information at specific junctures. These junctures and the timing of the junctures may be preset based on the treatment program. Alternatively, or additionally, the treatment center or care provider may have an access to the system account to input when a patient is entering various treatment stages and the timing junctures associated with each stage. As the patient enters approaches or leaves a juncture, the system may provide detail to the patient-adjacent person about what they can expect at that stage, for example about what they can expect from the patient at that stage. It may further provide detail about what is normal to expect, or what would be a cause for concern.

The information provided by the system may be customized to the user. In general, the system can cue how the patient-adjacent person might be feeling and give them input and suggestions. In one embodiment, the system may prompt the patient-adjacent person with questions such as “do you feel angry?” and can provide feedback based on the answer. For example, if the patient-adjacent person responds “Yes,” the system may explain that is normal and why. Conversely, if the patient-adjacent person responds “No,” the system can ask provide different feedback. Further, the system may provide alternative follow up questions or prompts based on the answers to more customize the feedback given to the patient-adjacent person.

With input by the treatment facility or provider, the system can cue up questions and feedback for the patient-adjacent person. The system can identify when the patient is going to experience something significant and proactively address the experience with the patient-adjacent person. For example, at some junctures, it may be likely that the patient is going to contact the patient-adjacent person. The system may indicate “This is what your loved one is about to experience, you can expect this kind of call, and that is very normal.” In another example, when a patient is about to be discharged, the system may contact the patient-adjacent person and say “your loved one is being discharged, here are ten things to think about to get your house ready.” The system provides predictive, anticipatory support to the patient-adjacent person.

In some embodiments, the system may provide daily content with meditations, positive feedback, and the like. The system may further provide linkage to therapy or therapeutic resources for the patient adjacent person. This may include, for example, counseling for skills development and to improve their own quality of life.

In some embodiments, the information and solutions available to a user may be tiered and the user may select the level of support they want from the system. For example, the system may provide, at different tiers, free information, books, digital content, support groups, and/or individual counseling.

In a simplified embodiment, a system and method is disclosed that provides a patient-adjacent person with passive guidance through a treatment process. The system can provide preset content that describes the treatment process, provides helpful tips, and provides description of and access to timely resources.

The system methodology may use a timeline having one or more segments (such as 30-day segments), each segment having topics and content directed to what happens during treatment at a time-period corresponding to the segment. Content may include books, articles, and the like. Content may further include in-person and/or virtual programs where patient-adjacent people can learn about addiction and the recovery process. Topic-based workshops (for example, healthy relationships, boundaries, etc.) may be offered throughout the timeline, during certain segments, or at certain junctures. In addition to, or as part of, the segments, weekly sessions may be offered to patient-adjacent people. These sessions may be tailored to junctures occurring during the segment or during the treatment process or may be open sessions to explore topics.

Content (whether text, image, video or combinations thereof) may be delivered to, or otherwise made available to, client devices via communications network. In some implementations, the output is posted on a website for view by the patient and/or patient-adjacent person, a mobile app interface, or other user interface that is maintained by a service provider. In other implementations, the content is delivered via email, SMS text notification, or push notifications from the mobile app. Content may be delivered, in some implementations, according to user identity or preferences stored in a data repository. For instance, delivering content may include determining a frequency of providing the supportive links based upon a preference of an identified patient-adjacent person, and generating the text summaries according to the preferred frequency of the identified patient-adjacent person.

In a further embodiment, the system and method may be customized for a military application. More specifically, the system and method may be customized for a service adjacent population, such as family or friends of military service members. Common deployment related mental health disorders for which service member may seek treatment include Acute Stress Disorder (ASD), Post Traumatic Stress Disorder (PTSD), Traumatic Brain Injury (TBI), survivors'guilt, depression, substance abuse, and others.

In such applications, the system and method may have a plurality of options including, for example, one for service adjacent persons of a service member wherein the applicable program the system is mapped to is based on time before deployment, during deployment, and/or after deployment. In a different option, the system may be mapped to a program addressing treatment of a service member who is wounded.

In some embodiments, the system and method may be used to facilitate a service adjacent person to be able to better monitor and refer service members for additional mental health services, and positively impact service member retention as well as positive home life.

The system and method may be used for guiding a person through any suitable program having identifiable junctures and information useful at one or more of such junctures. For example, the system and method may be used directly by a patient such as a situation where a patient is engaging in continuous care with poorly defined cognitive and psychological guideposts for how they should feel and think. In another embodiment, the system and method may be configured for use by employees in an employment assistance program.

For purposes of this disclosure, any system described herein may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a system or any portion thereof may be a minicomputer, mainframe computer, personal computer (e.g., desktop or laptop), tablet computer, embedded computer, mobile device (e.g., personal digital assistant (PDA) or smart phone) or other hand-held computing device, server (e.g., blade server or rack server), a network storage device, or any other suitable device or combination of devices and may vary in size, shape, performance, functionality, and price. A system may include volatile memory (e.g., random access memory (RAM)), one or more processing resources such as a central processing unit (CPU) or hardware or software control logic, ROM, and/or other types of nonvolatile memory (e.g., EPROM, EEPROM, etc.). A basic input/output system (BIOS) can be stored in the non-volatile memory (e.g., ROM), and may include basic routines facilitating communication of data and signals between components within the system. The volatile memory may additionally include a high-speed RAM, such as static RAM for caching data.

Additional components of a system may include one or more disk drives or one or more mass storage devices, one or more network ports for communicating with external devices as well as various input and output (I/O) devices, such as digital and analog general purpose I/O, a keyboard, a mouse, touchscreen and/or a video display. Mass storage devices may include, but are not limited to, a hard disk drive, floppy disk drive, CD-ROM drive, smart drive, flash drive, or other types of non-volatile data storage, a plurality of storage devices, a storage subsystem, or any combination of storage devices. A storage interface may be provided for interfacing with mass storage devices, for example, a storage subsystem. The storage interface may include any suitable interface technology, such as EIDE, ATA, SATA, and IEEE 1394. A system may include what is referred to as a user interface for interacting with the system, which may generally include a display, mouse or other cursor control device, keyboard, button, touchpad, touch screen, stylus, remote control (such as an infrared remote control), microphone, camera, video recorder, gesture systems (e.g., eye movement, head movement, etc.), speaker, LED, light, joystick, game pad, switch, buzzer, bell, and/or other user input/output device for communicating with one or more users or for entering information into the system. These and other devices for interacting with the system may be connected to the system through I/O device interface(s) via a system bus, but can be connected by other interfaces such as a parallel port, IEEE 1394 serial port, a game port, a USB port, an IR interface, etc. Output devices may include any type of device for presenting information to a user, including but not limited to, a computer monitor, flat-screen display, or other visual display, a printer, and/or speakers or any other device for providing information in audio form, such as a telephone, a plurality of output devices, or any combination of output devices.

A system may also include one or more buses operable to transmit communications between the various hardware components. A system bus may be any of several types of bus structure that can further interconnect, for example, to a memory bus (with or without a memory controller) and/or a peripheral bus (e.g., PCI, PCIe, AGP, LPC, I2C, SPI, USB, etc.) using any of a variety of commercially available bus architectures.

One or more programs or applications, such as a web browser and/or other executable applications, may be stored in one or more of the system data storage devices. Generally, programs may include routines, methods, data structures, other software components, etc., that perform particular tasks or implement particular abstract data types. Programs or applications may be loaded in part or in whole into a main memory or processor during execution by the processor. One or more processors may execute applications or programs to run systems or methods of the present disclosure, or portions thereof, stored as executable programs or program code in the memory, or received from the Internet or other network. Any commercial or freeware web browser or other application capable of retrieving content from a network and displaying pages or screens may be used. In some embodiments, a customized application may be used to access, display, and update information. A user may interact with the system, programs, and data stored thereon or accessible thereto using any one or more of the input and output devices described above.

A system of the present disclosure can operate in a networked environment using logical connections via a wired and/or wireless communications subsystem to one or more networks and/or other computers. Other computers can include, but are not limited to, workstations, servers, routers, personal computers, microprocessor-based entertainment appliances, peer devices, or other common network nodes, and may generally include many or all of the elements described above. Logical connections may include wired and/or wireless connectivity to a local area network (LAN), a wide area network (WAN), hotspot, a global communications network, such as the Internet, and so on. The system may be operable to communicate with wired and/or wireless devices or other processing entities using, for example, radio technologies, such as the IEEE 802.xx family of standards, and includes at least Wi-Fi (wireless fidelity), WiMax, and Bluetooth wireless technologies. Communications can be made via a predefined structure as with a conventional network or via an ad hoc communication between at least two devices.

Hardware and software components of the present disclosure, as discussed herein, may be integral portions of a single computer, server, controller, or message sign, or may be connected parts of a computer network. The hardware and software components may be located within a single location or, in other embodiments, portions of the hardware and software components may be divided among a plurality of locations and connected directly or through a global computer information network, such as the Internet. Accordingly, aspects of the various embodiments of the present disclosure can be practiced in distributed computing environments where certain tasks are performed by remote processing devices that are linked through a communications network. In such a distributed computing environment, program modules may be located in local and/or remote storage and/or memory systems.

As will be appreciated by one of skill in the art, the various embodiments of the present disclosure may be embodied as a method (including, for example, a computer-implemented process, a business process, and/or any other process), apparatus (including, for example, a system, machine, device, computer program product, and/or the like), or a combination of the foregoing. Accordingly, embodiments of the present disclosure may take the form of an entirely hardware embodiment, an entirely software embodiment (including firmware, middleware, microcode, hardware description languages, etc.), or an embodiment combining software and hardware aspects. Furthermore, embodiments of the present disclosure may take the form of a computer program product on a computer-readable medium or computer-readable storage medium, having computer-executable program code embodied in the medium, that define processes or methods described herein. A processor or processors may perform the necessary tasks defined by the computer-executable program code. Computer-executable program code for carrying out operations of embodiments of the present disclosure may be written in an object oriented, scripted or unscripted programming language such as Java, Perl, PHP, Visual Basic, Smalltalk, C++, or the like. However, the computer program code for carrying out operations of embodiments of the present disclosure may also be written in conventional procedural programming languages, such as the C programming language or similar programming languages. A code segment may represent a procedure, a function, a subprogram, a program, a routine, a subroutine, a module, an object, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the context of this document, a computer readable medium may be any medium that can contain, store, communicate, or transport the program for use by or in connection with the systems disclosed herein. The computer-executable program code may be transmitted using any appropriate medium, including but not limited to the Internet, optical fiber cable, radio frequency (RF) signals or other wireless signals, or other mediums. The computer readable medium may be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device. Computer-readable media includes, but is not to be confused with, computer-readable storage medium, which is intended to cover all physical, non-transitory, or similar embodiments of computer-readable media.

Various embodiments of the present disclosure may be described herein with reference to flowchart illustrations and/or block diagrams of methods, apparatus (systems), and computer program products. It is understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks. Alternatively, computer program implemented steps or acts may be combined with operator or human implemented steps or acts in order to carry out an embodiment of the invention.

Additionally, although a flowchart or block diagram may illustrate a method as comprising sequential steps or a process as having a particular order of operations, many of the steps or operations in the flowchart(s) or block diagram(s) illustrated herein can be performed in parallel or concurrently, and the flowchart(s) or block diagram(s) should be read in the context of the various embodiments of the present disclosure. In addition, the order of the method steps or process operations illustrated in a flowchart or block diagram may be rearranged for some embodiments. Similarly, a method or process illustrated in a flow chart or block diagram could have additional steps or operations not included therein or fewer steps or operations than those shown. Moreover, a method step may correspond to a method, a function, a procedure, a subroutine, a subprogram, etc.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A method for guiding a patient-adjacent person through a treatment process, the method comprising:
associating a patient-adjacent person with a patient;
assessing the treatment process that the patient is undergoing, wherein the treatment process includes segments, junctures, and/or milestones; and
providing content according to a patient-adjacent person program, wherein the program provides content to the patient-adjacent person;
wherein the patient-adjacent person program contextualizes the content based on at least one of the segments, junctures, and milestones.

2. The method of claim 1, and further comprising identifying a segment, juncture, or milestone, and delivering content based on the identified segment, juncture, or milestone.

3. The method of claim 2, wherein the content is contextualized based on the expected experience of or questions raised by the patient at the segment, juncture, or milestone.

4. The method of claim 3, wherein the content is delivered in advance of the patient reaching the segment, juncture, or milestone.

5. The method of claim 1, and further comprising prompting the patient-adjacent person for feedback on an emotional state, and adjusting the content based on the emotional state.

6. The method of claim 1, and further comprising iteratively realign the content based on experiences of the patient or the patient-adjacent person in the treatment process.

7. The method of claim 1, wherein the content includes clinical resources suitable for guiding the patient through treatment, wherein the clinical resources are modified based on information relating to the patient-adjacent person.

8. The method of claim 1, wherein the content is contextualized based on a machine learning model.

9. The method of claim 1, wherein providing content comprises delivering the content to a client device accessible by the patient adjacent person.

10. The method of claim 1, wherein the treatment process is related to behavioral health.

11. The method of claim 10, wherein the treatment process is associated with a substance abuse disorder.

12. A system for guiding a patient-adjacent person through a treatment process, the system comprising:
a processor; and
memory storing instructions which, when executed by the processor, cause the system to:
associate a patient-adjacent person with a patient;
assess the treatment process that the patient is undergoing, wherein the treatment process includes at least one of segments, junctures, and milestones; and
access a patient-adjacent person program based on the treatment process, wherein the program provides content to the patient-adjacent person;
wherein the patient-adjacent person program contextualizes the content based on at least one of the segments, junctures, and milestones.

13. The system of claim 12, wherein the content includes clinical resources suitable for treating the patient.

14. The system of claim 13, and, wherein the instructions, when executed by the processor, are configured to cause the system to deliver the content to a client device associated with the patient-adjacent person.

15. The system of claim 14, wherein the content is delivered at a time corresponding to one of the segments, junctures, and milestones.

16. The system of claim 12, wherein the content is based on at least one of similarly situated patients and similarly situated patient-adjacent persons.

17. The system of claim 16, and wherein the instructions further processor is further configured to develop content for the patient adjacent person program.

18. The system of claim 12, wherein the instructions, when executed by the processor, are configured to cause the system to identify a segment, juncture, or milestone, and delivering content based on the identified segment, juncture, or milestone.

19. The system of claim 18, wherein the content is contextualized based on the expected experience of or questions raised by the patient at the segment, juncture, or milestone.

20. The system of claim 19, and further comprising an artificial intelligence model, the model trained to modify program content based on at least one of the patient's and the patient-adjacent persons progress through the treatment process.
